(19) United States
(12) Patent Application Publication
(10) Pub. No.: US 2012/0108551 A1
(43) Pub. Date: May 3, 2012

(54) OPHTHALMIC COMPOSITIONS

(76) Inventors: Frank F. Molock, JR., Orange Park, FL (US); John C. Heaton, Atlantic Beach, FL (US)

(21) Appl. No.: 12/915,548

(22) Filed: Oct. 29, 2010

(51) Int. Cl. A61K 31/662; C07C 69/533; C07C 309/42; C07F 9/38; C07C 63/04; A61P 27/04; C07C 309/47; A61K 31/215; A61K 31/195; A61K 31/192; A61K 31/185; A61P 27/02; C07C 309/61; C07C 65/24

(52) U.S. Cl. 514/119; 562/55; 562/52; 560/1; 562/58; 562/76; 562/15; 562/23; 562/8; 562/405; 562/433; 562/465; 562/59; 514/529; 514/563; 514/567; 514/569; 514/568; 514/576; 514/577; 514/114; 514/141; 514/120; 514/129

(57) ABSTRACT

The present invention relates to compositions that may alleviate symptoms of ocular stress, as well as methods of their production, use, and storage compositions. The compositions comprise at least one ocular epithelial cell associating group and at least one hydrophilic group. In one embodiment the at least one ocular epithelial cell associating group and at least one hydrophilic group are substituents on a conjugated polyaromatic core.

The compositions may be used in ophthalmic compositions and ophthalmic devices.

### FIELD OF THE INVENTION

This invention relates to compositions that may alleviate symptoms of ocular stress, as well as methods of their production, use, and storage.

### BACKGROUND OF THE INVENTION

When the ocular environment becomes stressed it is often observed as a rapidly tear film break up time on contact lens wearers. In some cases ocular stress may be manifested in a break or thinning of the mucin layer. Stress to the ocular environment may cause the eyes to feel dry and irritated.

Several attempts have been made to alleviate the symptoms of ocular stress. For example, there are many over the counter eye drops aimed at lubricating the eye. These prior attempts use viscous solutions of hydrophilic polymers. However, they have met with little success since their residence time in the eye is so short.

Other formulations such as Systane and Hypotears and GenTeal provide formulations which, when combined with tears, form temporary crosslinks to form a network with a gel-like consistency to increase residence time and binds to the corneal surface. However, these solutions also do not provide the desired residence time.

Vital dyes have been used to assess if corneal and conjunctival cells are under stress and have been used as an important diagnostic tools in clinical ophthalmology for may surface eye disorders. The most commonly used dyes are fluoroscein, rose bengal and lissamine green b. However, the dyes are brightly colored at the concentrations used for diagnostic purposes, and can also sting upon instillation in the eye.

### SUMMARY OF THE INVENTION

The present invention relates to compositions comprising at least one ocular epithelial cell associating group and at least one hydrophilic group, having the formulae:

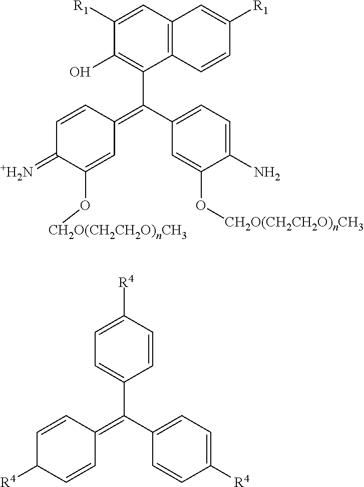

### Or

### DESCRIPTION OF THE FIGURES

FIGS. 1-4 are mass spectra for the compounds made in Examples 1-4.

FIGS. 5-8 are reactions schemes suitable for making artificial mucins of Formula II.

### DESCRIPTION OF THE INVENTION

The compounds of the present invention may be used a wide variety of ophthalmic compositions, including ophthalmic solutions, gels, ointments, dissolvable inserts and the like. Examples of known ophthalmic compositions include eye drops, eye washes, ocular gels and ointments and the like. The compositions of the present invention act as artificial mucins which when introduced into the ocular environment of a mammal displaying at least one area of mucin layer which is compromised or disrupted, fill said disrupted or compromise area.

The artificial mucin compounds of the present invention comprise at least two distinct groups: at least one ocular epithelial cell associating group and at least one hydrophilic group and have the formula:

(R)_{b}-Q-(R⁴)ₘ

wherein R is independently selected from the group consisting of sulfonates, phosphonates, carboxylates, ophthalmically compatible salts thereof;

R² and R³ are independently selected from H, and C₁₋₃ alkyls,

Q is a selected from conjugated polyaromatics having 2-3 aromatic rings and fused polyaromatics comprising 2-12 aromatic rings;

b is an integer between 1 and z; where z is the number of aromatic rings; in Q; m is an integer between 1 and z; and the sum of m+b is not greater than z;

R⁴ is selected from —NR²R³, —CH₂(CH₂CH₂O)ₙCH₃, —C₂₋₄ alkylene(X)ₚ where X is a hydrophilic monomer selected from hydrophilic (meth)acrylates and (meth)acrylamides, and p is an integer between 3-40; with the proviso that at least one and said composition has a log P between about −2 and 5.

In one embodiment Q is a fused polyaromatic having 2-6 aromatic rings, and in another a fused polyaromatic having 2-3 aromatic rings.

In another embodiment at least one of R is selected from the group consisting of sulfonates and ophthalmically compatible salts thereof.

In yet another embodiment, at least one R⁴ group of the artificial mucin comprises at least one C₂₋₄ alkylene(X)ₚ and p is an integer from 3-35. In yet another embodiment, the hydrophilic monomer X is selected from N,N-dimethyl acrylamide, 2-hydroxyethyl methacrylate, glycerol methacrylate, 2-hydroxyethyl methacrylamide, polyethyleneglycol monomethacrylate, methacrylic acid and acrylic acid, N-vinyl pyrrolidone, N-vinyl-N-methyl acetamide, N-vinyl-N-ethyl acetamide, N-vinyl-N-ethyl formamide, N-vinyl formamide, and combinations thereof.

In another embodiment the hydrophilic monomer is selected from the group consisting of N,N-dimethyl acrylamide, hydroxyethyl methacrylamide, N-vinyl pyrrolidone, N-vinyl-N-methyl acetamide, N-vinyl-N-ethyl acetamide, and combinations thereof.

The substituents on the artificial mucin are selected to provide the compound with a polarity measured by octanol-water partition coefficient, log P, of between about −2 to about 5 and in other embodiments between about −1 and 2.7 and in other embodiments is between about −0.7 and 1.5. The log P is the ratio of concentrations of un-ionized compound in water and octanol. The pH of the aqueous phase is adjusted such that the predominant form of the compound is un-ionized. The logarithm of the ratio of the concentrations of the un-ionized solute in the solvents is called log P and is expressed using the following equation:

Log P_{oct/water}=log([solute]_{octanol}/[solute]_{un-ionized water})

In another embodiment, the artificial mucins of the present invention have the formula:

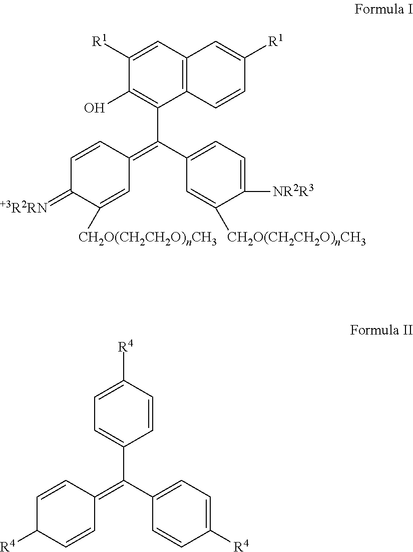

Wherein R₁ is independently selected from the group consisting of sulfonates, phosphonates, carboxylates, ophthalmically compatible salts thereof and combinations thereof,

R₂ and R₃ are independently selected from H, and C₁₋₃ alkyls,

R₄ is independently selected from C₁₋₃ alkyls and —CH₂(CH₂CH₂O)ₙCH₃, with the proviso that at least one R₄ must be —CH₂(CH₂CH₂O)ₙCH₃ and n is an integer 2-200, and in some embodiments from 2 to 100, in other embodiments from 2-10 in other embodiments still from 2-5.

The cations for the R₁ salts include ophthalmically compatible salts including sodium, potassium, calcium, lithium, ammonium and mixtures thereof. In one embodiment the salts are selected from sodium, potassium, calcium and mixtures thereof. In one embodiment, the artificial mucin of the present invention is a composition of Formula I having the structure:

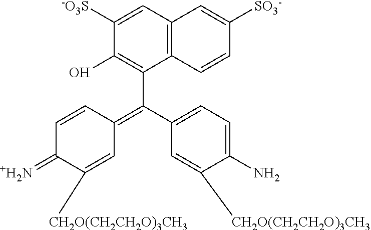

In another embodiment the artificial mucin is a composition of Formula II wherein

At least one R₄ is —CH₂(CH₂CH₂O)ₙCH₃ and the remaining R₄ are methyl or ethyl groups. In another embodiment the composition of Formula II one R₄ is —CH₂—O(CH₂CH₂O)₂CH₃ and the remaining R₄ are methyl. In further embodiments
two R₄ on the same N are —CH₂—O(CH₂CH₂O)₂CH₃ and the remaining R₄ are methyl,
one R₄ on each N is —CH₂—O(CH₂CH₂O)₂CH₃ and the remaining R₄ are methyl, and

### all R₄ are —CH₂—O(CH₂CH₂O)₂CH₃.

As used herein “ocular epithelial cells” are epithelial cells in the cornea or conjunctiva.

The artificial mucins of the present invention associate superficially with the surface of the epithelial cell. As used herein, “superficially” means that the artificial mucins, associate or bond with the surface of the cell only, and there is substantially no association or penetration through or into the cell wall. Examples of superficial association include temporary association or bonding, covalent bonding with chemical groups which are on or extend from the epithelial cell surface and combinations thereof and the like.

The artificial mucins of the present invention are capable of binding or associating with stressed, disrupted or damaged ocular epithelial cells.

In one embodiment the artificial mucin of the present invention comprises a residue from lissamine green, CI44090.

The artificial mucin compositions of the present invention further comprise at least one hydrophilic polyethylene oxide group. The hydrophilic polyethylene oxide groups extend from the epithelial associating group and attract water. In this way, the compositions of the present invention can enter a breach in the mucin layer of a stressed or injured eye. The epithelial cell associating group associates superficially with the epithelial cell and the hydrophilic polyethylene oxide group extends out to fill in the breach in the mucin layer and reestablish a continuous tear film. The hydrophilic group must be sufficiently hydrophilic so that the artificial mucin compound has a water solubility of at least 20%. The water solubility is determined by combining 20 wt % of the compound and 80 wt % borate buffered saline (pH=7.2-7.5), and heating the mixture with stirring overnight to 60° C. Solubility is determined by observing whether the material is dispersed and the solution is clear, although small amounts of impurities may cause small amounts of undissolved residue, or slight haziness to persist. Clarity may be measured via percent transmission. Suitable % transmission include 95% T for a 1 cm thick sample thickness.

The artificial mucins of Formula II of the present invention may be synthesized by reacting a leaving group terminated polyalkylene glycol of the desired molecular weight with n-C₁₋₃ alkyl aniline to form one of the polyalkylene glycol-substituted aniline rings. Suitable leaving groups include halides (Cl, Br, I and the like) and tosylates. Where artificial mucins having different substitutions on the aniline rings are desired, the aniline ring is formulated using a Vikmeier-Haak reaction and then reacted with another aniline compound via a Grignard reaction to form a bicyclic polyalkylene glycol substituted aniline structure. The second aniline compound may be alkyl substituted, such as N,N-dimethylaniline, or may be have a different polyalkylene glycol substitution.

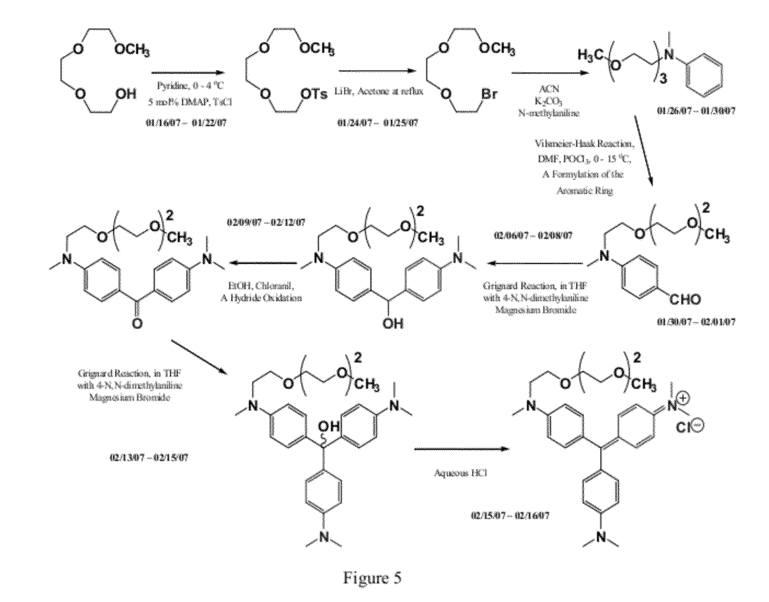

The bicyclic aniline compound is then subjected to a hydride oxidation in the presence of chloranil and a suitable solvent to form the bicyclic ketone, which is then subjected to a Grignard reaction with the desired aniline compound and a Grignard reagent. An example of this reaction scheme is shown in FIG. 5.

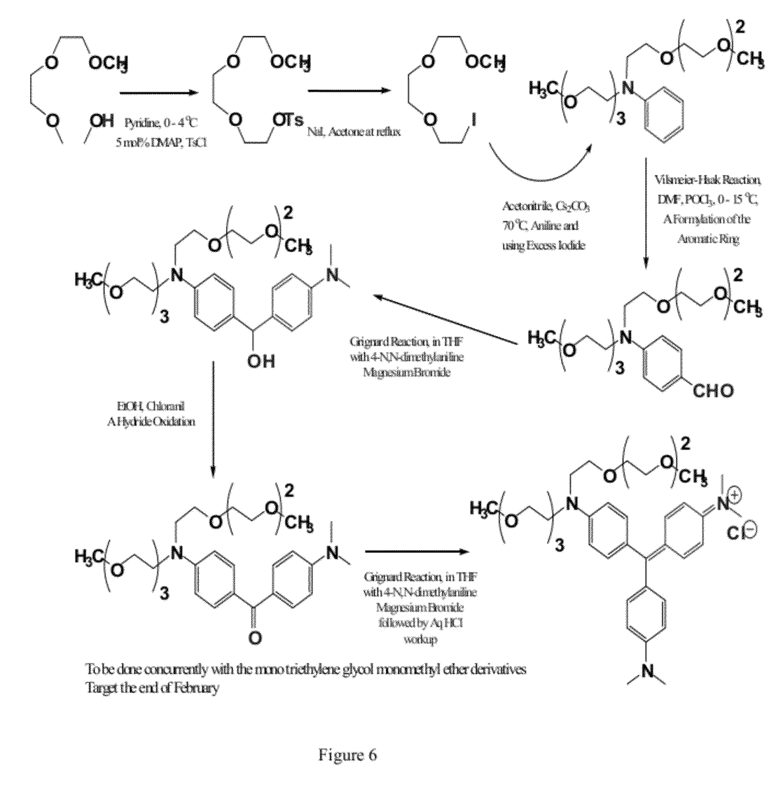

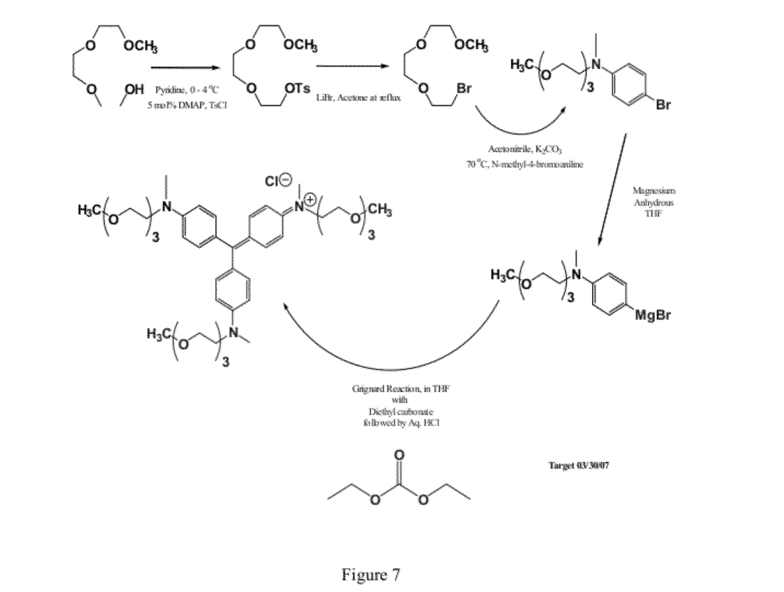

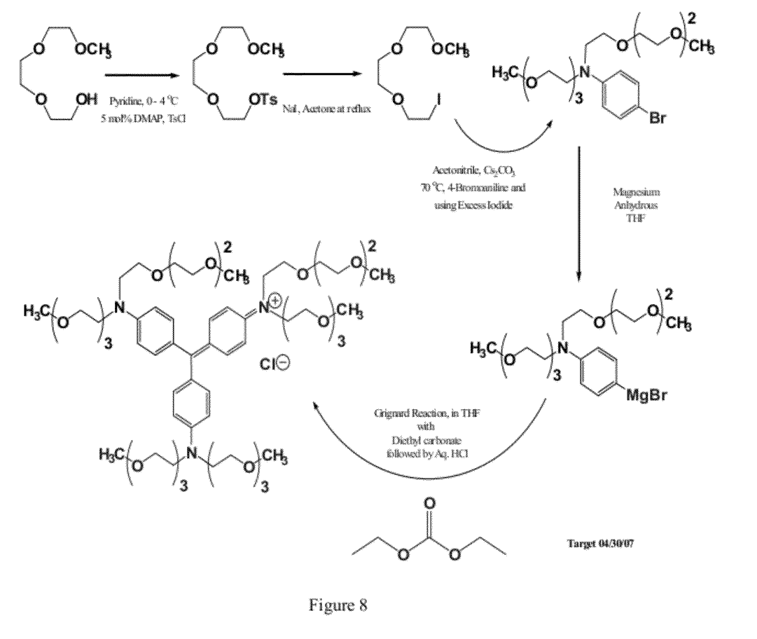

Alternate reaction schemes for artificial mucins of Formula II are shown in FIGS. 6-8. The artificial mucins of Formula I may be made by subjecting lissamine green (CI 44090) to Friedl Crafts alkylation with a halide substituted Polyalkylene glycol followed by purification.

The artificial mucin compositions of the present invention are suitable for direct application or instillation into the eye. Accordingly, the artificial mucin compositions and any formulations comprising them are free from compounds or agents which would be harmful to the ocular environment. For example, the artificial mucin compositions and formulations of the present invention are free from alkylating agents which are capable of intercalating between DNA.

The artificial mucin compositions may be included in a variety of ophthalmic formulations including ophthalmic solutions, gels, ointments and the like. Examples of ophthalmic formulations include eye drops, eye washes, as well as ophthalmic suspensions, gels and ointments and the like. The ophthalmic formulations may be instilled directly in the eye, or they may be used to clean and/or condition ophthalmic devices, such as contact lenses. In one embodiment of the present invention, the ophthalmic formulation is an ophthalmic solution.

The artificial mucin compositions may be present in ophthalmic formulations in amounts of about 1 to about 100 ppm, and in some embodiments between about 100 and about 10000 ppm. Ophthalmic formulations may also comprise mixtures of the artificial mucin compositions of the present invention.

The ophthalmic formulations of the present invention also have a pH of between about 6 and 8, and in some embodiments between about 6.5 and about 7.5. This allows the formulations of the present invention to be instilled directly in the eye, and to be used on or released from ophthalmic devices that are to be placed in the ocular environment.

The ophthalmic formulations of the present invention may comprise additional components such as, but not limited to carriers, pH adjusting agents, tonicity adjusting agents, buffering agents, active agents, lubricating agents, disinfecting agents, viscosity adjusting agents, surfactants and mixtures thereof. When the ophthalmic formulation is an ophthalmic solution, all components in the ophthalmic solution of the present invention should be water soluble. As used herein, water soluble means that the components, either alone or in combination with other components, do not form precipitates or gel particles visible to the human eye at the concentrations selected and across the temperatures and pH regimes common for manufacturing, sterilizing, storing and using the ophthalmic solution.

The pH of the ophthalmic formulation may be adjusted using acids and bases, such as mineral acids such as hydrochloric acid and bases such as sodium hydroxide.

The tonicity of the ophthalmic formulation may be adjusted by including tonicity adjusting agents. In some embodiments it is desirable for the ophthalmic formulation to be isotonic, or near isotonic with respect to normal, human tears. Suitable tonicity adjusting agents are known in the art and include alkali metal halides, phosphates, hydrogen phosphate and borates. Specific examples of tonicity adjusting agents include sodium chloride, potassium chloride, calcium chloride, magnesium chloride, zinc chloride, combinations thereof and the like.

The ophthalmic formulation may further comprise at least one buffering agent. Examples of suitable buffering agents include borate buffers, sulfate buffers, combinations thereof and the like. In one embodiment the buffering agent comprises borate buffer.

The ophthalmic formulation may also comprise at least one preservative. Any known preservative may be used. The preservative should not cause stinging or damage to the eye at use concentrations and should be inert with respect to the other composition components. Suitable preservatives include polymeric biguanides, polymeric quaternary ammonium compounds, chlorites, bisbiguanides, quaternary ammonium compounds, hydrogen peroxide, stabilized hydrogen peroxide and mixtures thereof.

One or more lubricating agents may also be included in the opththalmic formulation. Lubricating agents include water soluble cellulosic compounds, hyaluronic acid, and hyaluronic acid derivatives, chitosan, water soluble organic polymers, including water soluble polyurethanes, polyethylene glycols, polyamides, combinations thereof and the like. Specific examples of suitable lubricating agents include polyvinyl pyrrolidone, polyvinyl methylacetamide, hydroxypropyl methyl cellulose, glycerol, polyethylene glycols, mixtures thereof and the like. When a lubricating agent is used, it may be included in amounts up to about 5 weight %, and in some embodiments between about 100 ppm and about 2 weight %.

One or more active agent may also be incorporated into the ophthalmic formulation. A wide variety of therapeutic agents may be used. Suitable therapeutic agents include those that treat or target any part of the ocular environment, including the anterior and posterior sections of the eye and include pharmaceutical agents, vitamins, nutraceuticals combinations thereof and the like. Suitable classes of active agents include antihistamines, antibiotics, glaucoma medication, carbonic anhydrase inhibitors, anti-viral agents, anti-inflammatory agents, non-steroid anti-inflammatory drugs, antifungal drugs, anesthetic agents, miotics, mydriatics, immunosuppressive agents, antiparasitic drugs, anti-protozoal drugs, combinations thereof and the like. When active agents are included, they are included in an amount sufficient to produce the desired therapeutic result (a “therapeutically effective amount”). In one embodiment the ophthalmic formulation comprises at least one anti-inflammatory agent.

The ophthalmic formulation of the present invention may also include one or more surfactant. Suitable examples include poloxomer (poly(ethylene oxide)-b-poly(propylene oxide)-b-poly(ethylene oxide)) type surfactants which are commercially available from BASF and poloxamine type surfactants (non-ionic, tetrafunctional block copolymers based on ethylene oxide/propylene oxide, terminating in primary hydroxyl groups, commercially available from BASF, under the tradename Tetronic). A specific example is Pluronic F-147 and Tetronic 1304. Surfactants may be used in amounts up to about 5 weight %, and in some embodiments up to about 2 weight %.

Additionally, the ophthalmic formulation may comprise one or more viscosity adjusting agent or thickener. Suitable viscosity adjusting agents are known in the art and include polyvinyl alcohol, polyethylene glycols, guar gum, combinations thereof and the like. The viscosity adjusting agent may be used in amounts necessary to achieve the desired viscosity.

Ophthalmic solutions of the present invention may be formed by mixing the selected components with water. Other ophthalmic formulations may be formed by mixing the selected components with a suitable carrier.

These examples do not limit the invention. They are meant only to suggest a method of practicing the invention. Those knowledgeable in ophthalmics as well as other specialties may find other methods of practicing the invention. However, those methods are deemed to be within the scope of this invention.

The following abbreviations are used in the Examples.

### DMAP

EtOAc Ethyl acetate
TEGMME-OTs Triethylene glycol mono-methyl ether, mono-tosylate
TEGMME-Bromo 1-bromo-[(2-(2-(2-methoxyethoxy)ethoxy)ethane)]
TEGMME-Iodo 1-Iodo-[(2-(2-(2-methoxyethoxy)ethoxy)ethane)]

### Example 1

### Synthesis of N-methyl-N-[(2-(2-(2-methoxyethoxy)ethoxy)ethyl)]aniline

In a 3-neck, 100-mL round bottom flask, mounted within a Silvered Dewar set atop a magnetic stirring plate was placed a magnetic stir bar, 50 mL of dry tetrahydrofuran and (1.05 eq.), 2.48 g (23.1 mmol) of N-methyl-aniline (Sigma-Aldrich) that had been previously distilled in vacuo over zinc metal (Sigma-Aldrich).

To the Dewar was added 2-propanol and dry ice to reduce the temperature of the contents within the flask to −78° C. whilst stirring under N₂ atmosphere. Once equilibrated to −78° C., 10.2 mL (25.4 mmol) nBuLi/Hexane solution was added (from 2.5 M nBuLi in Hexane, Sigma-Aldrich). The mixture was stirred for 1 h giving the N-methyl-N-lithiated anilide salt; a pristine white precipitate in quantitative yield. To this dispersion was added 5.00 g (22.02 mmol) of 1-Bromo-[(2-(2-(2-methoxyethoxy)ethoxy)ethane)] (neat). The dry ice bath was removed and the flask and its contents were allowed to come to ambient temperature slowly over time (2-3 h) giving rise to an opaque turbid medium with a slightly yellow tint.

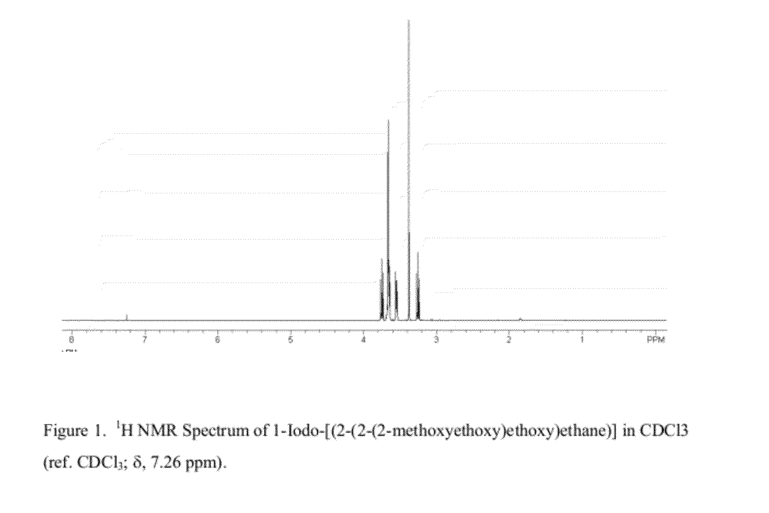

The reaction was quenched with several drops of 1 M HCl_{(aq)} and then poured into 100 mL of 100 mM sodium thiosulfate and extracted with 100 mL of chloroform. The organic layer is dried with sodium sulfate and the yellow oil is concentrated in vacuo. The oil was purified by flash chromatography on 60 Angstrom, 200-425 mesh, Davisil Grad 633 Silica-gel (Fisher), by gradient elution from 90/10 Hexs/EtOAc to 50/50 Hexs/EtOAc. The product was concentrated in vacuo by roto-evaporation giving 4.34 g in 78% yield. The TLC R_{f} value was 0.56 (50/50 Hexs/EtOAc on normal phase silica-gel). The material is used without further purification. The ¹H and ¹³C NMR Spectra and Electro-spray Mass Spectrum are attached as FIG. 1.

### Example 2

### Synthesis of 1-Bromo-[(2-(2-(2-methoxyethoxy)ethoxy)ethane)]

In a 3-neck, 250-mL round bottom flask, fitted with a West Condenser, heating mantle and voltage controller, a magnetic stirring plate and stir bar was placed (1 eq.), 9.15 g (28.7 mmol) TEGMME-OTs and dissolved with 100 mL of acetone (Sigma-Aldrich). Whilst stirring at room temperature (1.2 eq.) lithium bromide (Acros), 2.99 g (34.4 mmol) was added to the flask which dissolved with continued stirring. The flask was purged with N₂ and gently warmed to allow the acetone to reflux. Within a few minutes lithium tosylate began to precipitate from the medium. The reaction was allowed to reflux overnight and cooled to ambient in the morning.

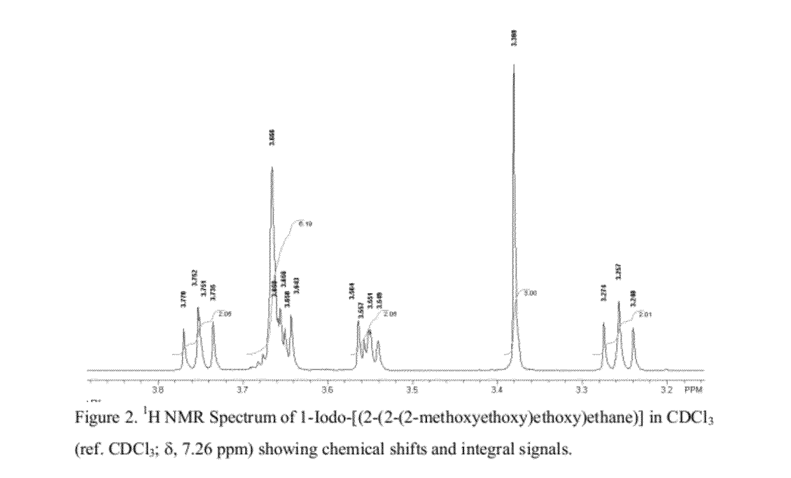

Lithium tosylate was removed by filtration and the acetone was stripped in vacuo using a roto-evaporator. The medium was taken up in 100 mL of chloroform and extracted with 100 mL, 100 mM aqueous sodium thiosulfate. The organic medium was dried with anhydrous sodium sulfate and the product was concentrated to a clear oil in vacuo giving 5.0 g of 1-bromo-[(2-(2-(2-methoxyethoxy)ethoxy)ethane)] (TEGMME-Bromo) in 77% yield. The TLC R_{f} value is 0.70 (50/50 Hexs/EtOAc on normal phase silica-gel). The material is used without further purification. The ¹H and ¹³C NMR Spectra and Electro-spray Mass Spectrum are attached as FIG. 2.

### Example 3

### Synthesis of 1-Iodo-[(2-(2-(2-methoxyethoxy)ethoxy)ethane)]

In a 3-neck, 250-mL round bottom flask, fitted with a West Condenser, heating mantle and voltage controller, a magnetic stirring plate and stir bar was placed (1 eq.), 20.5 g (64.5 mmol) TEGMME-OTs and dissolved with 100 mL of acetone (Sigma-Aldrich). Whilst stirring at room temperature (1.2 eq.) sodium iodide (Sigma-Aldrich), 11.6 g (77.4 mmol) was added to the flask which dissolved with continued stirring. The flask was purged with N₂ and gently warmed to allow the acetone to reflux. Within a few minutes sodium tosylate began to precipitate from the medium. The reaction was allowed to reflux overnight and cooled to ambient in the morning.

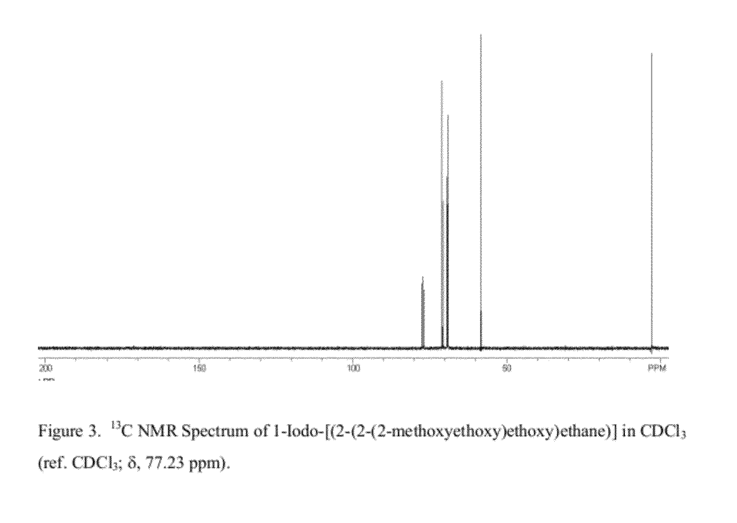

Sodium tosylate was removed by filtration and the acetone was stripped in vacuo using a roto-evaporator. The medium was taken up in 100 mL of chloroform and extracted with 100 mL, 100 mM aqueous sodium thiosulfate. The organic medium was dried with anhydrous sodium sulfate and the product was concentrated to a clear oil (slightly yellow-tint) in vacuo giving 16.9 g of 1-Iodo-[(2-(2-(2-methoxyethoxy)ethoxy)ethane)] (TEGMME-Iodo) in 96% yield. The TLC R_{f} value is 0.64 (50/50 Hexs/EtOAc on normal phase silica-gel). The material was used without further purification and tends to yellow over time. The elemental analysis (found: C, 30.7; H, 5.6; O, 17.0; and I, 46.0; C₇H₁₅IO₃, requires: C, 30.67; H, 5.52; I, 46.30; O, 17.51). The ¹H and ¹³C NMR Spectra and Electro-spray Mass Spectrum are attached as FIG. 3.

### Example 4

### Synthesis of Triethylene glycol mono-methyl ether, mono-tosylate[1-(2-(2-(2-methoxyethoxy)ethoxy)ethyl)]-p-toluenesulfonate

In a 2-liter, 2-piece, flanged, reaction kettle (three neck upper-flange and a lower jacketed reaction flask) fitted with a Friedrich's Condenser, mechanical stirring and a thermostatted liquid circulator (for controlling reactor temperatures) was added (1 eq.), 41.25 g of 97% TEGMME (or 40.00 g, 243.6 mmol, Fluka) and was dissolved with 300 mL of pyridine (Fluka). Next was added (1.2 eq.), 55.78 g (292.3 mmol) of p-toluenesulfonyl chloride (Acros). The reaction was catalyzed 5 mol % DMAP (1.80 g, 14.6 mmol, Sigma-Aldrich). The reagents were stirred under N₂ atmosphere at 25° C. for 6 hours. The temperature of the circulated liquid about the reactor jacket was reduced to 10° C. and the reaction medium was stirred overnight. In the morning pyridinium hydrochloride had precipitated from the reaction medium and this was further facilitated by reducing the circulated liquid to −15° C. and gently stirring the reaction for an additional 4 h.

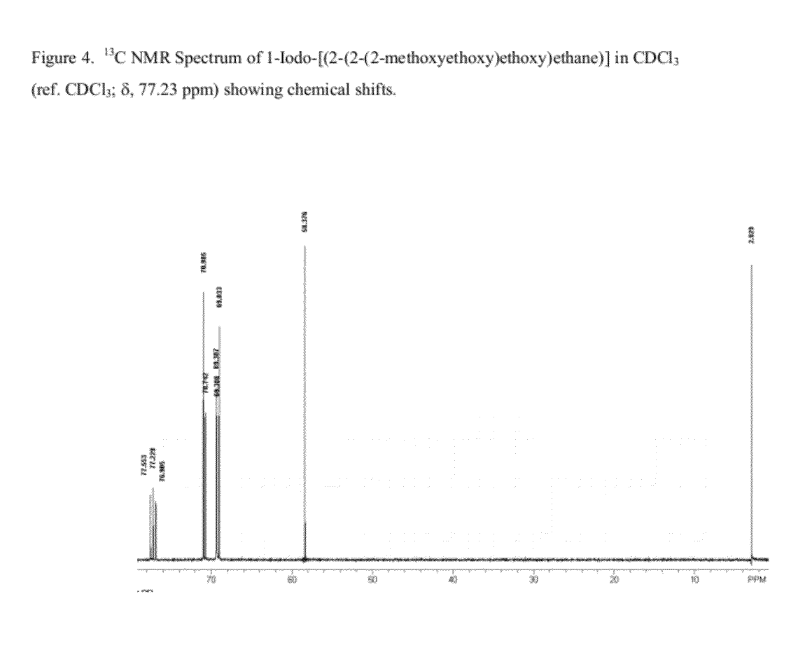

The pyridinium hydrochloride precipitate was removed by filtration and to the reaction medium was added 600 mL of 50/50 v/v concentrated hydrochloric acid and deionized water. The aqueous medium was extracted with chloroform and the organic layers combined and were dried with anhydrous sodium sulfate and then concentrated down to an oil in vacuo on a roto-evaporator. The oil was purified by flash chromatography on 60 Angstrom, 200-425 mesh, Davisil Grad 633 Silica-gel (Fisher), eluting with 50/50 hexanes/ethyl acetate. The product was concentrated in vacuo by roto-evaporation giving 43.17 g of Triethylene glycol mono-methyl ether, mono-tosylate (TEGMME-OTs) in 56% yield. The TLC R_{f} value is 0.56 (50/50 Hexs/EtOAc on normal phase silica-gel). The ¹H and ¹³C NMR Spectra and Electro-spray Mass Spectrum are attached as FIG. 4.

We claim:

1. A composition comprising
(R)_{b}-Q-(R⁴)ₘ
wherein R is independently selected from the group consisting of sulfonates, phosphonates, carboxylates, ophthalmically compatible salts thereof;
R² and R³ are independently selected from H, and C₁₋₃ alkyls,
Q is a selected from conjugated polyaromatics having 2-3 aromatic rings and fused polyaromatics comprising 2-12 aromatic rings;
b is an integer between 1 and z; where z is the number of aromatic rings; in Q; m is an integer between 1 and z; and the sum of m+b is not greater than z;
R⁴ is selected from —NR²R³, —CH₂(CH₂CH₂O)ₙCH₃, —C₂₋₄ alkylene(X)ₚ where X is a hydrophilic monomer selected from hydrophilic (meth)acrylates and (meth)acrylamides, and p is an integer between 3-40; with the proviso that at least one and said composition has a log P between about −2 and 5.

2. The composition of claim 1 wherein Q is a fused polyaromatic having 2-6 aromatic rings.

3. The composition of claim 1 wherein Q is a fused polyaromatic having 2-3 aromatic rings.

4. The composition of claim 1 wherein at least one of R is selected from the group consisting of sulfonates, phosphonates, carboxylates, ophthalmically compatible salts thereof.

5. The composition of claim 1 wherein p is an integer from 3-35.

6. The composition of claim 1 wherein said composition has a log P between about −1 and 2.7.

7. The composition of claim 1 wherein said composition has a log P between about −0.7 and 1.5.

8. The composition of claim 1 wherein said hydrophilic monomer is selected from the group consisting of N,N-dimethyl acrylamide, 2-hydroxyethyl methacrylate, glycerol methacrylate, 2-hydroxyethyl methacrylamide, polyethyleneglycol monomethacrylate, methacrylic acid and acrylic acid, N-vinyl pyrrolidone, N-vinyl-N-methyl acetamide, N-vinyl-N-ethyl acetamide, N-vinyl-N-ethyl formamide, N-vinyl formamide, and combinations thereof.

9. The composition of claim 1 wherein said hydrophilic monomer is selected from the group consisting of N,N-dimethyl acrylamide, hydroxyethyl methacrylamide, N-vinyl pyrrolidone, N-vinyl-N-methyl acetamide, N-vinyl-N-ethyl acetamide, and combinations thereof.

10. A composition comprising at least one artificial mucin of formula I or II 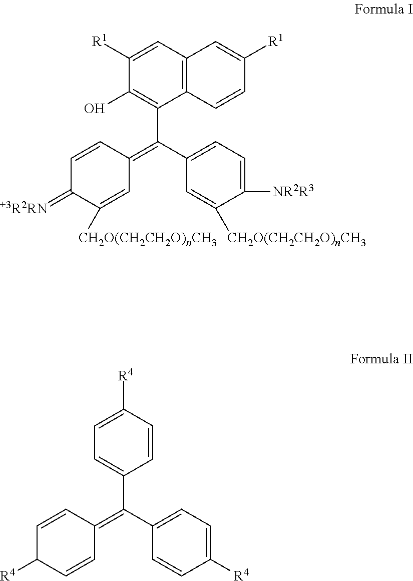
Wherein R¹ is independently selected from the group consisting of sulfonates, phosphonates, carboxylates, ophthalmically compatible salts thereof and combinations thereof,
R² and R³ are independently selected from H, and C₁₋₃ alkyls,
R⁴ is independently selected from C₁₋₃ alkyls and —CH₂(CH₂CH₂O)ₙCH₃, with the proviso that at least one R⁴ must be —CH₂(CH₂CH₂O)ₙCH₃ and
n is an integer 3-200.

11. The composition of claim 10 wherein said artificial mucin comprises

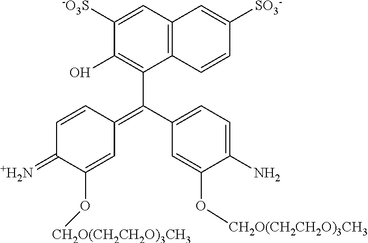

12. The composition of claim 10 wherein n is an integer of 3 to 100.

13. The composition of claim 10 wherein R¹ is further comprises at least one cation selected from ophthalmically compatible salts.

14. The composition of claim 13 wherein said ophthalmically compatible cation is independently selected from the group consisting of sodium, potassium, calcium, lithium, ammonium and mixtures thereof.

15. The composition of claim 13 wherein said ophthalmically compatible cation is independently selected from the group consisting of sodium, potassium, calcium and mixtures thereof.

16. The composition of claim 10 wherein R₁ comprises a sulfonate salt.

17. The composition of claim 10 wherein the artificial mucin is a composition of Formula II and at least one R₄ is —CH₂(CH₂CH₂O)ₙCH₃ and the remaining R⁴ are methyl or ethyl groups.

18. The composition of claim 10 wherein the artificial mucin is a composition of Formula II and one R⁴ is —CH₂—O(CH₂CH₂O)₂CH₃ and the remaining R⁴ are methyl.

19. The composition of claim 10 wherein the artificial mucin is a composition of Formula II and two R₄ on the same N are —CH₂—O(CH₂CH₂O)₂CH₃ and the remaining R⁴ are methyl.

20. The composition of claim 10 wherein the artificial mucin is a composition of Formula II and one R₄ on each N is —CH₂—O(CH₂CH₂O)₂CH₃ and the remaining R⁴ are methyl.

21. The composition of claim 10 wherein the artificial mucin is a composition of Formula II and all R⁴ are —CH₂—O(CH₂CH₂O)₂CH₃.

22. The composition of claim 10 wherein said composition is soluble in borate buffered saline at 60° C.

23. A method for mitigating the symptom of dry eye in a patent comprising administering to said patient's eyes a composition of claim 1 or 10.

24. An ophthalmic solution comprising a composition of claim 1 or 10.

25. A method comprising introducing into the ocular environment of a mammal displaying at least one area of mucin layer which is compromised or disrupted, at least one composition of claim 1 or 10 in an amount sufficient to fill said area of compromised or disrupted mucin layer.
